(19) United States
(12) Patent Application Publication
(10) Pub. No.: US 2026/0232485 A1
(43) Pub. Date: Aug. 13, 2026

(54) WEARABLE HEATING DEVICE FOR LUMBAR AND ABDOMINAL REGION

(71) Applicant: Shenzhen iKeepfit Technology Co., Ltd., Shenzhen (CN)

(72) Inventors: LIJUN WAN, Shenzhen (CN); YE SHEN, Shenzhen (CN)

(21) Appl. No.: 19/632,063

(22) Filed: Mar. 27, 2026

(30) Foreign Application Priority Data
Jan. 22, 2026 (CN) 202610088388.8

(51) Int. Cl. A61F 7/02; A61F 7/00

(52) U.S. Cl. CPC A61F 7/02; A61F 7/007; A61F 2007/0022; A61F 2007/0027; A61F 2007/0078; A61F 2007/0093; A61F 2007/0295

(57) ABSTRACT

Provided is a wearable heating device for lumbar and abdominal region, which includes a wearing body, a heating element, a control motherboard, a control module, and a first battery box and a second battery box. The wearing body is fixed to an abdomen of a human body. The heating element is provided on one side of the wearing body that contacts the abdomen. The first battery box and the second battery box both have built-in batteries, and the two batteries are electrically connected to the control motherboard. The present disclosure uses dual batteries for independent or collaborative power supply, which not only extends the battery life, but also improves power supply stability and usage safety.

### CROSS-REFERENCE TO RELATED APPLICATIONS

This application claims priority to the Chinese patent application with application number of CN202610088388.8 and entitled with “wearable heating device for lumbar and abdominal region”, submitted to CNIPA on January 22, 2026. Which is hereby incorporated by reference in its entirety.

### TECHNICAL FIELD

The present disclosure relates to the field of hot compress equipment technologies, and in particular, to a wearable heating device for lumbar and abdominal region.

### BACKGROUND

Wearable heating device for lumbar and abdominal region is a hot compress product used to relieve menstrual discomfort in women, usually powered by a built-in lithium battery. At present, the wearable heating device for lumbar and abdominal region on the market generally adopts a single battery compartment design. Although the structure is simple, there are obvious defects in practical use: on the one hand, due to the concentrated arrangement of batteries on one side of the main body, the center of gravity of the whole machine is offset, causing unilateral force and imbalance during wearing, which can easily slide or compress local skin, affecting the comfort and stability of wearing. On the other hand, the short power supply time of a single battery affects the overall battery life of the wearable heating device for lumbar and abdominal region.

### SUMMARY

The present disclosure aims to provide a wearable heating device for lumbar and abdominal region to solve the technical problems of low wearing comfort and short power supply time caused by the single battery design of the wearable heating device for lumbar and abdominal region.

The present disclosure provides a wearable heating device for lumbar and abdominal region, including:

a wearing body, configured to fix the wearable heating device for lumbar and abdominal region on an abdomen of a human body;

a heating element, which is provided on one side of the wearing body that contacts the abdomen of the human body;

a control motherboard, which is electrically connected to the heating element;

a control module, which is integrated on the control motherboard and configured to receive control instruction to control an operation of the heating element;

a first battery box and a second battery box that are both provided on one side of the wearing body facing away from the heating element, the first battery box and the second battery box are provided on two sides of the heating element along a length direction of the wearing body; the first battery box includes a first box body and a first battery, and the first battery is electrically connected to the control motherboard; the second battery box includes a second box body and a second battery, and the second battery is electrically connected to the control motherboard.

From the above technical solution, it can be seen that the embodiments of the present disclosure have at least the following advantages and positive effects.

The wearable heating device for lumbar and abdominal region provided by the embodiments of the present disclosure is designed with two battery boxes (first battery box and second battery box). On the one hand, dual battery redundant power supply significantly extends the battery life: by providing the first battery box and the second battery box on the back of the wearing body, the two sets of batteries can independently or cooperatively power the heating element, effectively solving the problem of short usage time of traditional single battery products and meeting user’s long-term therapy needs. On the other hand, the two battery boxes design can automatically switch or complement power supply when one set of batteries is low or faulty, avoiding interruption of therapy due to power outages, while reducing the risk of overheating caused by high load operation of single battery and improving safety of use. Finally, the design of two battery boxes avoids the problems of unilateral force and uneven wearing, thereby enhancing the overall comfort of the wearable heating device for lumbar and abdominal region. Finally, the heating element is provided on the side of the wearing body that contacts the abdomen, and the two battery boxes are provided on the side of the wearing body that faces away from the abdomen, it is distributed at left and right of the human body, which can effectively adjust the balance of the center of gravity distribution, avoid being heavy in the front and light in the back, and enhance the balance and comfort of users wearing the wearable heating device for lumbar and abdominal region. In summary, the wearable heating device for lumbar and abdominal region of the present disclosure balances functionality, safety, and comfort, and has good practical value and market prospects.

### BRIEF DESCRIPTION OF DRAWINGS

In order to illustrate the technical solutions of the embodiments of the present disclosure or of the prior art more clearly, the following drawings are briefly described as required in the context of the embodiments or the prior art. Obviously, the following drawings illustrate only some of the embodiments of the present disclosure. Other relevant drawings may be obtained on the basis of the shown structures of these drawings without any creative effort by those skilled in the art.

FIG. 1 is a schematic diagram of an overall structure of a wearable heating device for lumbar and abdominal region according to an embodiment of the present disclosure.

FIG. 2 is an exploded view of the wearable heating device for lumbar and abdominal region described in FIG. 1.

FIG. 3 is an enlarged view of a control motherboard in the wearable heating device for lumbar and abdominal region shown in FIG. 2.

FIG. 4 is an enlarged view of a first battery box in the wearable heating device for lumbar and abdominal region shown in FIG. 2.

FIG. 5 is a schematic diagram of a cross-sectional structure along A-A direction (i.e. a longitudinal centerline of the wearing body) in FIG. 1.

FIG. 6 is an exploded view of the wearable heating device for lumbar and abdominal region according to another embodiment of the present disclosure.

Numeral reference: 1- wearable heating device for lumbar and abdominal region; 10- wearing body; 11- lining layer; 12- insulation layer; 13- fabric layer; 14- wiring channel; 15- first installation position; 16- second installation position; 20- heating element; 30- control motherboard; 31- first sub board; 32- second sub board; 40- control module; 50- first battery box; 51- first box body; 52- first battery; 53- LED indicator light; 54- observation window; 55- uniform light film; 56- charging interface; 60- second battery box; 61- second box body; 62- second battery; 70- button; 71- first button; 72- second button; 80- flexible conductive structure; 81- first electrical contact point; 82- second electrical contact point.

### DESCRIPTION OF EMBODIMENTS

Typical embodiments reflecting features and advantages of the present disclosure will be described in detail in the following description. It should be understood that the present disclosure may admit to various modifications in different embodiments without departing from the scope of the present disclosure. The descriptions and drawings therein are substantially illustrative in nature rather than restrictive of the present disclosure.

Furthermore, the related terms “first”, and “second” are used for descriptive purposes only and are not to be understood as indicating or implying relative importance or implicitly specifying the number of technical features indicated. Thus, features defined as “first” or “second” may explicitly or implicitly include one or more of such features. In the description of the present application, the term “a plurality of” means two or more unless otherwise expressly specified.

In the description of the present application, it should be noted that terms such as “installed,” “provided,” and “connected” should be broadly understood unless otherwise expressly defined and limited. For example, a connection may be a fixed connection, a detachable connection, or an integral connection; may be a mechanical connection or an electrical connection; may be a direct connection or an indirect connection through an intermediate medium; and may be communication between interiors of two components. Those of ordinary skill in the art may understand the specific meanings of the above terms in the present disclosure based on practical contexts

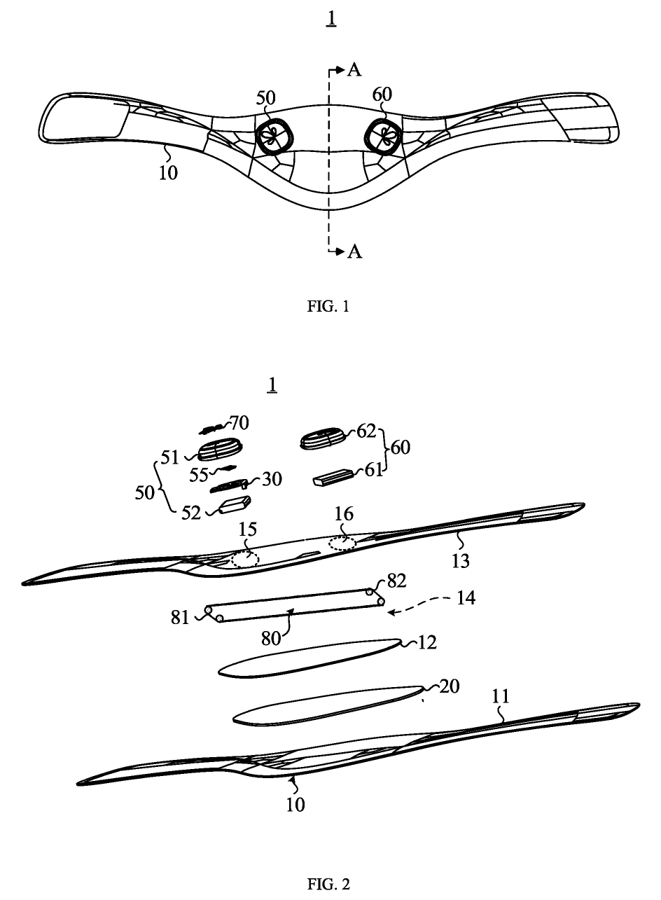

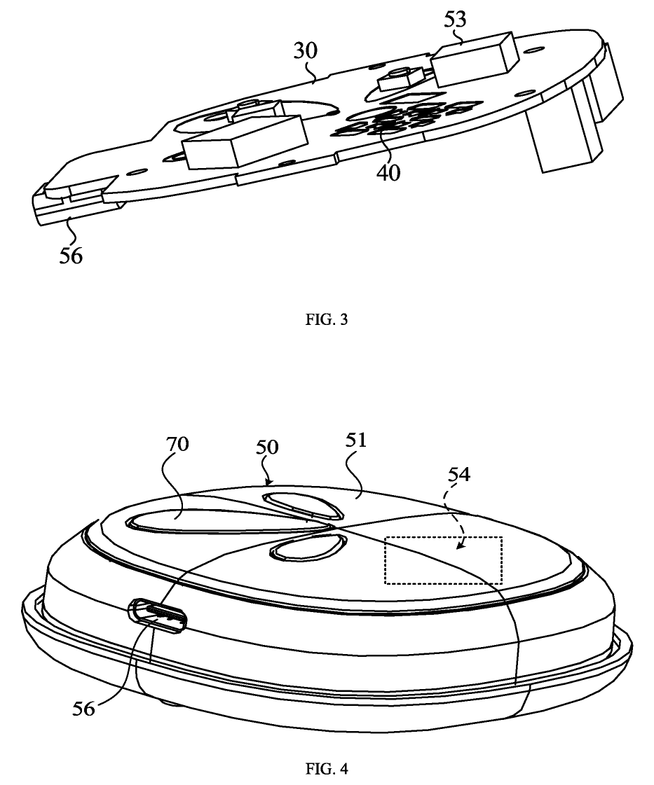

Referring to FIGS. 1-3, the present disclosure provides a comfortable wearable heating device for lumbar and abdominal region 1, including a wearing body 10, a heating element 20, a control motherboard 30, a control module 40, a first battery box 50, and a second battery box 60. Where the wearing body 10 is configured to fix the wearable heating device for lumbar and abdominal region 1 to a human abdomen, and the heating element 20 is provided on one side of the wearing body 10 that contacts the human abdomen, fitting closely to the human abdomen to provide thermal therapy. The control motherboard 30 serves as a control center for the entire wearable heating device for lumbar and abdominal region 1 and is provided on the wearing body 10 or the first battery box 50 or the second battery box 60. The control motherboard 30 is electrically connected to the heating element 20. The control module 40 is integrated onto the control motherboard 30 and is configured to receive an instruction to adjust an operation of the heating element 20. The first battery box 50 and the second battery box 60 are both provided on one side of the wearing body 10 facing away from the heating element 20. The first battery box 50 and the second battery box 60 are provided on two sides of the heating element 20 along a length direction of the wearing body 10. The first battery box 50 includes a first box body 51 and a first battery 52 accommodated in the first box body 51. The first battery 52 is electrically connected to the control motherboard 30. The second battery box 60 includes a second box body 61 and a second battery 62 accommodated inside the second box body 61. The second battery 62 is electrically connected to the control motherboard 30.

It can be understood that the heating element 20 here is broadly defined, referring to any component or structure that generates heat through power supply. In this application, due to a flat design of the entire wearable heating device for lumbar and abdominal region 1 to fit the curves of the human body, and the heating element 20 needs to fit the abdomen of the human body, it needs to be processed into a sheet shape to make the entire wearable heating device for lumbar and abdominal region 1 thinner and lighter. Besides that, the first battery 52 and the second battery 62 here can independently or collaboratively supply power to the heating element 20. Moreover, the number of battery boxes provided in this application is at least two, and the number of two or more can also achieve the effect of extending battery life and improving wearing balance and comfort.

The wearable heating device for lumbar and abdominal region 1 provided by the present disclosure, through the design of two battery boxes (first battery box 50 and second battery box 60), on the one hand, provides redundant power supply for the two batteries, significantly extending the battery life: by providing the first battery box 50 and the second battery box 60 on a back of the wearing body 10, the two sets of batteries can independently or cooperatively power the heating element 20, effectively solving the problem of short usage time of traditional single battery products and meeting the long-term physical therapy needs of users. On the other hand, the two battery boxes design can automatically switch or complement power supply when one set of batteries is low or faulty, avoiding interruption of therapy due to power outages, while reducing the risk of overheating caused by high load operation of single battery and improving safety of use. Finally, the design of two battery boxes avoids the problems of unilateral force and uneven wearing, thereby improving the comfort of wearing the entire wearable heating device for lumbar and abdominal region 1. Finally, the heating element 20 is provided on one side of the wearing body that contacts the abdomen, and the two battery boxes are provided on the side of the wearing body that faces away from the abdomen, which are distributed at the left and right of the human body, which can effectively adjust the balance of the center of gravity distribution, avoid being heavy in the front and light in the back, and improve the balance and comfort of users wearing the wearable heating device for lumbar and abdominal region 1. In summary, the wearable heating device for lumbar and abdominal region 1 of the present disclosure balances functionality, safety, and comfort, and has good practical value and market prospects.

In an implementation mode, referring to FIGS. 1 and 2, positions of the first battery box 50 and the second battery box 60 are symmetrically distributed about a longitudinal centerline of the wearing body 10 (i.e., the A-A section line). The positions of the first battery box 50 and the second battery box 60 are symmetrically distributed about the longitudinal centerline of the heating element 20, where the longitudinal centerline of the wearing body 10 and the longitudinal centerline of the heating element 20 merge into one. This design enables the user to adjust the heating element 20 to fit the abdomen after wearing the wearable heating device for lumbar and abdominal region 1. The position of the first battery box 50 and the second battery box 60 is symmetrical relative to the human body. On the premise of ensuring the efficient hot compress physiotherapy of the heating element 20, the symmetrical design of the two battery boxes is also ensured, to improve the balance and comfort of users when wearing wearable heating device for lumbar and abdominal region 1. In an implementation mode, the positions of the two battery boxes do not need to be symmetrically arranged about the longitudinal centerline, and the user can adjust the circumferential position of the wearing body 10 relative to the human body to adjust it to the left-right symmetry of the two battery boxes.

In order to further enhance the wearing balance and comfort of wearable heating device for lumbar and abdominal region 1, the weight difference between the left and right sides of the wearable heating device for lumbar and abdominal region 1 in the wearing state does not exceed 5%. That is, within an allowable range of weight error, the position weight of the first battery box 50 and the position weight of the second battery box 60 are symmetrically arranged relative to the longitudinal center of the wearing body 10, and the wearing balance and comfort of the wearable heating device for lumbar and abdominal region can be maintained without any adjustment by the user. In order to achieve equal weight of the two battery boxes, the first box body 51 and the second box body 61 have relative external dimensions and structures, and the model, capacity, and weight of the first battery 52 and the second battery 62 are the same. Of course, in other embodiments, the weight of the box bodies corresponding to the two battery boxes and the batteries may not be equal, and weight balance can be achieved by balancing the left and right weights. Finally, the weight of the two boxes may not be equal, and users can also adjust themselves to a comfortable and balanced wearing position.

In an implementation mode,, referring to FIGS. 2-4, the control motherboard 30 is encapsulated within the first box body 51. The wearable heating device for lumbar and abdominal region 1 further includes a button 70, with one end of the button 70 electrically connected to the control motherboard 30 and the other end extending from the first box body 51. The operation of the button 70 drives the control module 40 to control the operation of the heating element 20. Here, the button 70 can be implemented through movable buttons or touchscreen touch. It should be noted that in other embodiments, without providing the button 70, a user can wirelessly connect to the control motherboard 30 through a mobile Application, (APP) client or remote control. Operating the mobile phone or remote control can also drive the control module 40 to control the operation of the heating element 20. On the other hand, here, the first box body 51 provides a stable support and an installation space for the button 70. In an implementation mode, the button 70 can also be placed on the second box body 61 or the wearing body 10, and connected to the control motherboard 30 through wires or wireless communication.

In an implementation mode,, referring to FIGS. 2-4, the first box body 51 and/or the second box body 61 (at least one of the two boxes) are provided with light-emitting diode, (LED) indicator lights 53 electrically connected to the control motherboard 30. The box body provided on a light emission path of the LED indicator lights 53 is provided with a window 54 for the light emitted by the LED indicator lights 53 to pass through. The LED indicator lights 53 distinguish battery levels of the first battery 52 and the second battery 62 through different colors or flashing frequencies. For example, the common flashing colors of LED indicator lights 53 correspond to battery mode: LED indicator lights 53 are always on, and green indicates a high battery working state. The LED indicator lights 53 are constantly on, orange indicates low battery; the LED indicator lights 53 flash slowly in red to indicate charging is in progress. The LED indicator lights 53 are off, indicating that the wearable heating device for lumbar and abdominal region 1 is turned off or not working. It should be noted that the shutdown may be caused by the battery being depleted, or it may be directly controlled by the button 70 to turn on or off the device. It should be understood that in this application, the corresponding colors of the LED indicator lights 53 for different modes of the battery or device are not limited, and the number of modes is not limited. Besides that, in other embodiments, the setting of the LED indicator lights 53 is not limited. The wearable heating device for lumbar and abdominal region 1 can indicate the battery level mode by setting a buzzer alarm prompt module, or directly display the battery level value on a display screen for a user to intuitively obtain the high and low battery levels. Furthermore, the light emitting surface of the LED indicator lights 53 in this application is covered with a uniform light film 55 to enhance the luminous brightness. In other embodiments, the uniform light film 55 may not be provided.

In an implementation mode,, referring to FIGS. 2-4, the first battery 52 and the second battery 62 are rechargeable batteries, and the first battery 52 and the second battery 62 are in the same charge circuit. The first box body 51 is provided with a charging interface 56 electrically connected to the control motherboard 30, and one end of the charging interface 56 is exposed outside the first box body 51 for external charging power supply, allowing the first battery 52 and the second battery 62 to be charged simultaneously. It should be noted that in other embodiments, the charging interface 56 may not be provided, and the two rechargeable batteries can be taken out and charged before being assembled into the corresponding box for use; or, in an implementation mode, the charging interface 56 is not provided, and the first battery 52 or the second battery 62 can be selected as a dry battery, disposable battery, and directly replaced with a new one by replacing one set.

In an implementation mode,, referring to FIGS. 2 and 5, the wearing body 10 includes an inner layer 11, an insulation layer 12, and a fabric layer 13 arranged layer by layer. The heating element 20 is sandwiched between the inner layer 11 and the insulation layer 12, and a wiring channel 14 is provided between the insulation layer 12 and the fabric layer 13. The control motherboard 30 and the second battery 62 are electrically connected through a flexible conductive structure 80 built into the wiring channel 14. It can be understood that the flexible conductive structure 80 includes but is not limited to conductive wires or flexible circuit boards, and the flexible conductive structure 80 needs to bend and stretch with the wearing process of the wearing body 10. It should be noted that the insulation layer 12 not only slows down the heat dissipation process of the heating element 20, but also isolates the heating element 20 from the flexible conductive structure 80, thereby preventing the heating element 20 from damaging the flexible conductive structure 80 during the heating process, and protecting the flexible conductive structure 80. In an implementation mode, the wiring channel 14 and the flexible conductive structure 80 are not provided, and the control motherboard 30 and the second battery 62 can also be connected wirelessly, remotely controlled or controlled by a mobile app to control the electrical continuity between the two. In this case, if the heat dissipation of the heating element 20 is not considered, the setting of the insulation layer 12 is not limited.

In an implementation mode,, referring to FIGS. 2 and 5, the flexible conductive structure 80 forms a first electrical contact point 81 at one end of the wiring channel 14 and a second electrical contact point 82 at the other end of the wiring channel 14. The first electrical contact point 81 is provided at a first installation position 15 of the wearing body 10 configured to install the first box body 51, and the second electrical contact point 82 is provided at a second installation position 16 of the wearing body 10 configured to install the second box body 61. The control motherboard 30 is electrically connected to the first electrical contact point 81, and the second battery 62 is electrically connected to the second electrical contact point 82. Of course, in an implementation mode, there is no need to provide the first electrical contact point 81 or the second electrical contact point 82. Two ends of the flexible conductive structure 80 can also be electrically connected to the control board 30 and the second battery 62 through other structural means.

In an implementation mode,, the first box body 51 is detachably installed at the first installation position 15; and/or the second box body 61 can be disassembled and installed at the second installation position 16. Detachable installation makes it convenient for a user to replace the battery box, especially when the battery box needs to be charged or repaired, the user does not need to replace the entire wearable heating device for lumbar and abdominal region 1. In an implementation mode, two sets of conductive pillars with positive and negative electrodes can be used for the two electrical contact points. The two sets of conductive pillars are exposed and pass through the fabric layer 13 to form corresponding installation positions for the corresponding box body to be installed in alignment. The control motherboard 30 is electrically connected to the first electrical contact point 81 through a set of conductive pillars with positive and negative electrodes passing through the first box body 51, and the second battery 62 is electrically connected to the second electrical contact point 82 through a set of conductive pillars with positive and negative electrodes passing through the second box body 61. Furthermore, the first box body 51 can be magnetically aligned and detachably provided at the first installation position 15, and the second box body 61 can be magnetically aligned and detachably provided at the second installation position 16. It should be noted that, firstly, in this application, there is no limitation on whether the two boxes can be disassembled and provided in the corresponding installation positions. Secondly, the implementation mode of detachable installation of the two boxes in corresponding installation positions is not limited, and is not limited to magnetic attraction, snap fasteners, adhesives, and other methods.

Wearable heating device for lumbar and abdominal region 1 in embodiment 2

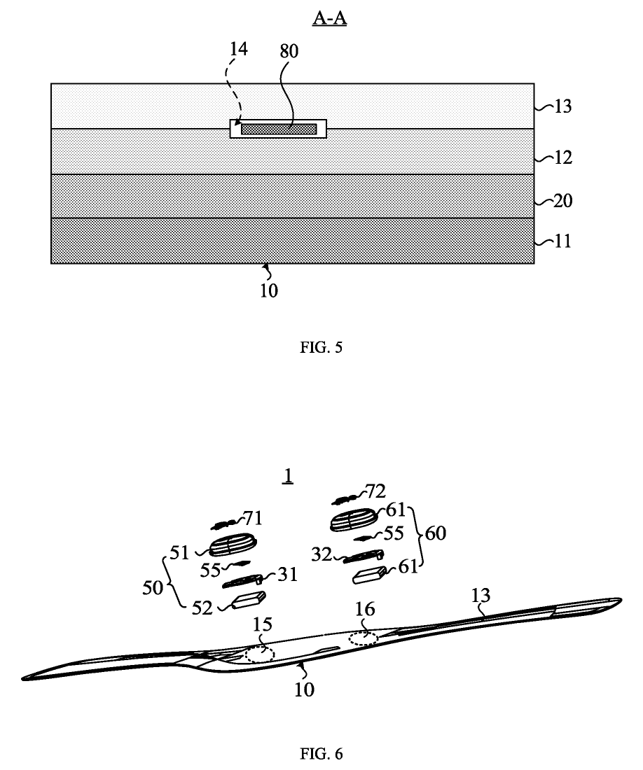

Referring to FIGS. 2, 4, and 6, the structure of the wearable heating device for lumbar and abdominal region 1 in this embodiment is basically the same as that of wearable heating device for lumbar and abdominal region 1 in the first embodiment. The difference is that wearable heating device for lumbar and abdominal region 1 in the first embodiment controls two batteries to supply power to the heating element 20 simultaneously through one control motherboard 30, and wearable heating device for lumbar and abdominal region 1 in this embodiment controls two batteries to supply power to the heating element 20 separately through two control motherboards 30. Specifically, the control motherboard 30 includes a first sub board 31 enclosed in a first box body 51 and a second sub board 32 enclosed in a second box body 61, both first sub board 31 and second sub board 32 are integrated with the control module 40. The wearable heating device for lumbar and abdominal region 1 further includes a first button 71 and a second button 72. One end of the first button 71 is electrically connected to the first sub board 31, and the other end extends out of the first box body 51. One end of the second button 72 is electrically connected to the second sub board 32, and the other end extends out of the second box body 61. The first battery 52 and the second battery 62 are connected in parallel to the control circuit, and the first button 71 and the second button 72 are operated to drive the corresponding control module 40 to independently control the heating element 20. When the user wears the wearable heating device for lumbar and abdominal region 1 of this embodiment, the first button 71 and the second button 72 are provided on two sides of the abdomen, rendering it convenient for the user to operate with two hands. It should be noted that the first button 71 and the first sub board 31 work together to independently control the first battery box 50 to supply power to the heating element 20. The second button 72 and the second sub board 32 work together to independently control the second battery box 60 to supply power to the heating element 20. Do not interfere with each other, if one part encounters a problem, the other part can continue to supply power. The two battery boxes are controlled separately, and the user can open only one side or two sides at the same time according to personal needs to meet more user needs.

Although the present disclosure has been described with reference to several typical embodiments, it should be understood that the terminology used is illustrative and exemplary rather than restrictive. Since the present disclosure can be implemented in various forms without departing from its spirit or essence, it should be understood that the above-described embodiments are not limited by any foregoing details but should be broadly interpreted within the spirit and scope defined by the appended claims. Therefore, all changes and modifications falling within the scope of the claims or equivalents thereof should be covered by the appended claims.

What is claimed is:

1. A wearable heating device for lumbar and abdominal region, comprising:
a wearing body, configured to fix the wearable heating device for lumbar and abdominal region on an abdomen of a human body;
a heating element, which is provided on one side of the wearing body that contacts the abdomen of the human body;
a control motherboard, which is electrically connected to the heating element;
a control module, which is integrated on the control motherboard and configured to receive control instruction to control an operation of the heating element;
a first battery box and a second battery box that are both provided on one side of the wearing body facing away from the heating element, the first battery box and the second battery box are provided on two sides of the heating element along a length direction of the wearing body;
the first battery box comprises a first box body and a first battery, and the first battery is electrically connected to the control motherboard;
the second battery box comprises a second box body and a second battery, and the second battery is electrically connected to the control motherboard.

2. The wearable heating device for lumbar and abdominal region according to claim 1, wherein a position of the first battery box and a position of the second battery box are symmetrically distributed about a longitudinal centerline of the wearing body.

3. The wearable heating device for lumbar and abdominal region according to claim 2, wherein a weight deviation between left and right sides of the wearable heating device for lumbar and abdominal region in a wearing state does not exceed 5%.

4. The wearable heating device for lumbar and abdominal region according to claim 1, wherein the control motherboard is encapsulated in the first box body, and the wearable heating device for lumbar and abdominal region further comprises a button, one end of the button is electrically connected to the control motherboard, and the other end extends out of the first box body, and the button is operated to drive the control module to control the operation of the heating element.

5. The wearable heating device for lumbar and abdominal region according to claim 4, wherein the first box body and/or the second box body are provided with LED indicator lights electrically connected to the control motherboard, and the box body provided on a light emission path of the LED indicator lights is provided with a window for light emitted by the LED indicator lights to pass through;
the LED indicator lights distinguish power levels of the first battery and the second battery through different colors or flashing frequencies.

6. The wearable heating device for lumbar and abdominal region according to claim 4, wherein the first battery and the second battery are rechargeable batteries, and the first battery and the second battery are in the same charge circuit;
the first box body is provided with a charging interface electrically connected to the control motherboard, and one end of the charging interface is exposed outside the first box body to connect to a charging power source, and the charging power source charges the first battery and the second battery simultaneously.

7. The wearable heating device for lumbar and abdominal region according to claim 1, wherein the wearing body comprises an inner layer, an insulation layer, and a fabric layer that are arranged layer by layer,
the heating element is sandwiched between the inner layer and the insulation layer, and a wiring channel is provided between the fabric layer and the insulation layer;
the control motherboard and the second battery are electrically connected through a flexible conductive structure built into the wiring channel.

8. The wearable heating device for lumbar and abdominal region according to claim 7, wherein the flexible conductive structure forms a first electrical contact point at one end of the wiring channel and a second electrical contact point at the other end of the wiring channel;
the first electrical contact point is provided at a first installation position of the wearing body configured to install the first box body, and the second electrical contact point is provided at a second installation position of the wearing body configured to install the second box body;
the control motherboard is electrically connected to the first electrical contact point, and the second battery is electrically connected to the second electrical contact point.

9. The wearable heating device for lumbar and abdominal region according to claim 8, wherein the first box body is detachable and provided in the first installation position.

10. The wearable heating device for lumbar and abdominal region according to claim 9, wherein the second box body is detachable and provided in the second installation position.

11. The wearable heating device for lumbar and abdominal region according to claim 1, wherein the control motherboard comprises a first sub board packaged in the first box body and a second sub board packaged in the second box body, both of first sub board and the second sub board are integrated with the control module;
the wearable heating device for lumbar and abdominal region further comprises a first button and a second button, wherein one end of the first button is electrically connected to the first sub board, and the other end extends out of the first box body;
one end of the second button is electrically connected to the second sub board, and the other end extends out of the second box body;
the first battery and the second battery are connected in parallel to the control circuit, and the first button and the second button are operated to drive the corresponding control module to individually control the heating element.
